(19) United States
(12) Patent Application Publication
(10) Pub. No.: US 2025/0169858 A1
(43) Pub. Date: May 29, 2025

(54) BONE SETTING DEVICE

(71) Applicant: Orit YAHALOM, Ganei Tikva (IL)

(72) Inventor: Orit YAHALOM, Ganei Tikva (IL)

(21) Appl. No.: 18/840,626

(22) PCT Filed: Feb. 24, 2024
(86) PCT No.: PCT/IB2023/051713

§ 371 Date: Aug. 22, 2024

Related U.S. Application Data
(60) Provisional application No. 63/314,271, filed on Feb. 25, 2022.

(51) Int. Cl. A61B 17/68

(52) U.S. Cl. CPC A61B 17/68; A61B 2017/681

(57) ABSTRACT

A bone fracture fixation device 10 for setting a fractured bone, the device comprising at least two sleeve portions, wherein a first sleeve portion 12 having measures to surround above 180 degrees of the bone, and the one or more additional sleeve portions 14 complement the first sleeve portion to thereby fully or essentially fully surround the fractured bone.

### FIELD OF THE INVENTION

The presently disclosed subject matter is in the field of skeletal health care, in particular bone fracture repair.

### BACKGROUND OF THE INVENTION

Bone fractures are typically set using an external cast external splint; or using a surgical procedure with rod(s), pin(s) or plate(s) fastened to the bone.

European Patent No. 0295041 discloses an internal fixation apparatus for bone fractures that has a framework structure made up of, three channel bars and two tension rings, each of the tension rings having at least one buckling structure and one fastening structure so that it may be divided into two pieces and form a closed ring around the fractured bone. The edges of the channel bars and tension rings have teeth formed thereon for engaging with the bone so as to fulfil stable fixation. The directional arrangement of the teeth makes it possible to apply an adequate pressure to the fractured site by the patient's own body weight and muscle construction. The apparatus is sui-table for fixing various kinds of bone fractures and beneficial to an even healing of the bone fractures and an im-proved strength of the healed bone.

Chinese Patent Application No. 103976784 discloses a customized orthopedic internal fixation support. The support comprises an A-side support and a B-side support which are engaged with a skeleton surface, wherein a positioning hole A is formed in one end of the A-side support, a permanent hole is formed in the other end of the A-side support, and a set screw A is arranged on the top of the A-side support; a stop pin is arranged at one end of the B-side support, and a locking screw is arranged at the other end of the B-side support, and a set screw B is arranged on the top of the B-side support; a single-sided support is adopted for a plane fracture, the structure of the single-sided support is the same as the flattened or deformed A-side support or B-side support.

US Patent Application No. 2004/0087955 discloses a device for the reduction and stabilization of broken bone parts, such as those deriving from a fracture, that is particularly suitable for veterinary use. The device comprises a tubular band as the main element, to be applied around the bone at the point of fracture or discontinuity, that is made of a metallic material and provided with a longitudinal aperture, equipped with multiple substantially pointed protrusions, distributed along its inner surface, for gripping the said broken bone parts. The tubular band is malleable to be wrapped tightly around the broken bone parts with the use of flexible cerclage elements, which are secured transversally around said tubular band and housed in external transverse grooves.

US Patent Application No. 2012/0029102 discloses systems for bone fracture repair. One system includes a biocompatible putty that may be packed about a bone fracture to provide full loadbearing capabilities within days. The disclosed putties create an osteoconductive scaffold for bone regeneration and degrade over time to harmless 5 resorbable byproducts. Further disclosed are fixation devices for contacting an endosteal wall of an intramedullary (IM) canal of a fractured bone.

### SUMMARY OF THE INVENTION

The presently disclosed subject matter relates to a device for setting a fractured bone, the device is configured to be inserted adjacent the fractured bone, to surround the fractured bone and fix it in place.

It is an objective of the invention to provide such a device that does not require drilling a screw into the bone in order to fixate the device.

It is a further object of the invention to provide a device that can be feasibly inserted to about the fractured bone despite the confined space due to the muscle tissues and ligaments around the fractured bone.

It is a further objective to allow bone metabolism while the device is in use.

Yet another objective of the invention is to stabilize the fractured bone with minimal contact between the device and the bone.

It is a further object of the invention to provide a device that will promote healing of the fracture by supplying bone growth factors and other therapeutic agents.

It is another object of the invention to provide a method for fixating a fractured bone by implementing the device of the invention.

In a first aspect, the present invention provides a bone fracture fixation device for setting a fractured bone, the device comprising:

a sleeve having measures to fully or essentially fully surround a segment of the fractured bone including the fracture comprising:

at least two sleeve portions, wherein a first sleeve portion having measures to surround above 180 degrees, and a second sleeve portion and optionally one or more additional sleeve portions complementing the first sleeve portion to thereby fully or essentially fully surround the fractured bone when the device is in use.

In one or more embodiments, the first sleeve portion surrounds more than 210 degrees, more than 230 degrees, more than 250 degrees, or more than 270 degrees of the fractured bone when the device is in use.

In one or more embodiments, the first sleeve portion surrounds at least 270 degrees of the fractured bone when the device is in use.

In one or more embodiments, the device comprising a first and a second sleeve portions, wherein the first and second sleeve portions surround 360 degrees about the fractured bone when the device is in use.

In one or more embodiments, the sleeve further comprising a plurality of protrusions attached to or extending from an inner wall of the sleeve to press on the fractured bone and fix the bone in place when the device is in use.

In one or more embodiments, the protrusions have sharp or blunt tips.

In one or more embodiments, the protrusions are dispersed along the entire inner wall of the sleeve.

In one or more embodiments, the protrusions are dispersed on the two or more portions of the sleeve.

In one or more embodiments, the sleeve comprises two orifices through which the fractured bone passes when the device is in use and the protrusions are dispersed in proximity to each of the orifices.

In one or more embodiments, a central portion of the inner wall of the sleeve that is proximal to the fracture is free of protrusion.

In one or more embodiments, the protrusions define a space between the bone and the sleeve.

In one or more embodiments, the protrusions are made of a rigid or a semi-rigid material being same or different than the material of the sleeve.

In one or more embodiments, the sleeve portions comprise a plurality of apertures to allow passage of nutrients and/or cells therethrough to assist in expediting healing of the fractured bone.

In one or more embodiments, the combined area of the plurality of apertures consists between 20 to 60% of the area of the sleeve.

In one or more embodiments, the sleeve portions comprise an inter-sleeve portion locking mechanism to allow closing the sleeve.

In one or more embodiments, the locking mechanism comprises one or more of screws, straps, wires, zip ties, fasteners, flanges, cerclage or a combination thereof.

In one or more embodiments, the locking mechanism is configured to allow closing the sleeve to a given diameter.

In one or more embodiments, the locking mechanism comprises cooperating adjacent edges of the sleeve portions to allow overlap thereof to thereby allow adjustment of the diameter of the sleeve.

In one or more embodiments, the mechanism comprises a diameter adjustment fixing fastener comprising an adjustable strap/wire/tie to set the sleeve diameter; or through-sleeve fasteners configured to attach overlapping sleeve areas.

In one or more embodiments, one or more of the sleeve portions and/or protrusions comprise bone growth factors, bone growth hormones, bone growth minerals (e.g., calcium), bone cells, and/or stem cells, that can be released to the bone and assist in expediting healing.

In one or more embodiments, the device further comprising a film disposed in the inner face of the one or more sleeve portions, the film comprising bone growth factors, bone growth hormones, bone growth minerals (e.g., calcium), bone cells, and/or stem cells, that can be released to the bone and assist in expediting healing.

In another aspect the invention provides a method of fixating a fractured bone of a human or non-human patient, the method comprising:
(a) providing access for a bone fracture fixation device to the fractured bone, the bone fracture fixation device being defined by any one of the preceding claims;
(b) placing a first sleeve portion at least partially around, the fractured bone;
(c) placing a second sleeve portion and optionally one or more additional sleeve portions around the fractured bone, so as to fully or essentially fully surround at least a portion of the fractured bone; and
(d) fastening the sleeve portions to each other.

In one or more embodiments, the first sleeve portion is dimensioned to surround above 180 degrees of the fractured bone and is placed prior to placing the second sleeve portions and optionally the one or more additional sleeve portions, so as to allow full surrounding of the fractured bone.

In one or more embodiments, the first sleeve portion is dimensioned to surround above 180 degrees of the fractured bone and is placed underneath the fractured bone.

In one or more embodiments, the method further comprising adjusting the diameter of the resultant sleeve to a given diameter.

In one or more embodiments, step (d) entails adjusting the diameter of the resultant sleeve so as to interface at least some of the protrusions with the fractured bone.

In one or more embodiments, step (d) entails using a locking mechanism configured to allow fastening using one or more of screws, straps, wires, zip ties, fasteners, flanges, cerclage or a combination thereof.

In one or more embodiments, step (d) entails overlapping adjacent edges of the sleeve portions to thereby allow adjustment of the diameter of the sleeve and fixing thereof.

In one or more embodiments, after step (d), the method further comprises removing the device from around the fractured bone.

In one or more embodiments, step (a) comprises surgical cutting tissue adjacent the bone fracture.

In one or more embodiments, the at least two sleeve portions are predesigned and custom-made to adapt the dimensions of the fractured bone.

In one or more embodiments, the dimensions are the diameter of the fractured bone.

In one or more embodiments, the fractured bone is of a non-human animal.

In one or more embodiments, the device is for use in veterinary purposes.

In one or more embodiments, step (b) comprises placing the first sleeve portion underneath the fractured bone.

In one or more embodiments, step (b) comprises placing the first sleeve portion so that it is located below the fractured bone (i.e., distally from an incision made for allowing access to the fractured bone).

In one or more embodiments, step (b) comprises placing the second sleeve portion so that it is located above the fractured bone (i.e., proximally with respect to an incision made for allowing access to the fractured bone).

In one or more embodiments, the method further comprising placing a film underneath the sleeve portions, wherein the film comprising bone growth factors, bone growth hormones, bone growth minerals (e.g., calcium), bone cells, and/or stem cells, that can be released to the bone and assist in expediting healing.

In one or more embodiments, the film is placed underneath the sleeve portions before or after placing the sleeve portions around the fractured bone.

### BRIEF DESCRIPTION OF DRAWINGS

The invention may be more clearly understood upon reading of the following detailed description of non-limiting exemplary embodiments thereof, with reference to the following drawings, in which:

FIG. 1 is a perspective exploded view of a bone fracture repair device in accordance with one example of the presently disclosed subject matter.

FIG. 2 is a perspective view of the bone fracture repair device of FIG. 1.

FIG. 3 is a front perspective view of the bone fracture repair device of FIG. 1, along with an enlarged area thereof.

FIG. 4 is an end view of the bone fracture repair device of FIG. 1, along with an enlarged area thereof.

FIG. 5A is a perspective view of the device of FIG. 1 when a first sleeve portion thereof wraps a fractured bone.

FIG. 5B is a perspective view of the device of FIG. 1 when the first and second sleeve portions fully surround the fractured bone.

### DETAILED DESCRIPTION OF EMBODIMENTS

The invention may be more clearly understood upon reading of the following detailed description of non-limiting exemplary embodiments thereof, with reference to the drawings.

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

Illustrative embodiments of the invention are described below. In the

interest of clarity, not all features/components of an actual implementation are necessarily described.

The present invention pertains to a bone fracture fixation device for setting a fractured bone, the device is devised of a sleeve configured to fully surround or essentially fully surround the fractured bone when the device is in use.

The term “fractured bone” may relate to any type of fracture of a bone of a vertebrate organism. The vertebrate organism may include without being limited thereto, to humans, primates (e.g. monkeys), canines (e.g. dogs), horses, felines (e.g. cats), birds and ruminants (e.g. cows, sheep, camels). The types of bone that are relevant to this invention are generally longitudinal bones or radial bones which may include without being limited thereto, femur, radius, humerus, vertebrae and ribs. The types of fractures which may be fixated by the device and method of the invention include without being limited thereto to hairline, greenstick, oblique (displaced or non-displaced), linear, transverse, comminuted, spiral or segmental.

The term “fully surround the fractured bone” refers to surrounding the periphery around the portion of the bone adjacent to the fracture. It should not be interpreted as necessarily surrounding the entire length of the bone, but rather as at least partially surrounding the fractured area of the bone.

The term “essentially fully surround the fractured bone” refers to surrounding above 80%, 90%, 95% or 99% of the periphery around the portion of the bone adjacent to the fracture.

The sleeve comprises at least two sleeve portions, which when coupled to each other fully surround or wrap the fracture. In an embodiment of the invention, the device includes a first sleeve portion that is configured to surround above 180 degrees.

In an exemplary embodiment, the device includes two sleeve portions, which when coupled to each other fully surround the fracture.

In an embodiment of the invention, the sleeve portions are equal in length. In an alternative embodiment, the sleeve portions have different lengths.

In an embodiment of the invention, the device includes a second sleeve portion that complements the first sleeve portion. The complementing portions may be designed such that they do not have to be exactly fitting to each other in order to connect and form the sleeve. The portions may be slightly translated one with respect to the other allowing some freedom to the operator to personalize and adapt the final structure of the sleeve to the individual fractured bone being treated. This can be achieved for example by large slots in the locking mechanism of each portion through which fasteners are inserted to connect the two portions together.

In an embodiment of the invention, one or more additional sleeve portions are further contemplated such that together, all sleeve portions fully surround or essentially fully surround the fractured bone when the device is in use.

As illustrated below, the first sleeve portion may be referred to as a major sleeve portion, or the larger portion out of the two portions, as it surrounds more than 180 degrees of the fractured bone when the device is in use, or alternatively-it defines an arc of more than 180 degrees of a given diameter. For example, the first sleeve portion surrounds more than 190 degrees, more than 210 degrees, more than 230 degrees, more than 250 degrees, more than 270 degrees, more than 300 or more than 330 degrees of the fractured bone when the device is in use; or defining an arc of more than 190 degrees, more than 210 degrees, more than 230 degrees, more than 250 degrees, more than 270 degrees, more than 300 or more than 330 degrees of a given diameter. In one embodiment, the first sleeve portion surrounds at least 270 degrees of the fractured bone when the device is in use or defining an arc of at least 270 degrees a given diameter. In one embodiment, the first sleeve portion surrounds at least 300 degrees of the fractured bone when the device is in use; or defining an arc of at least 300 degrees of given diameter. In some embodiments the second portion of the sleeve, being the smaller portion completes the first portion being the larger portion such that the combination of the two portions surrounds 360 degrees or close to 360 degrees about the fractured bone. The smaller portion may be defined as defining an arc which completes the arc of the major portion to 360 degrees or close to 360 degrees For example, in some embodiments the major portion of the sleeve surrounds 210 degrees, and the smaller portion of the sleeve surrounds 150 degrees about the fractured bone, respectively (when the diameter of the bone is equal or smaller than the diameter of the arc defined by each of the portions. In some embodiments the first portion of the sleeve surrounds 270 degrees and the second portion of the sleeve surrounds 90 degrees about the fractured bone, respectively (when the diameter of the bone is equal or smaller than the diameter of the arc defined by each of the portions). The given diameter of the sleeve defined by the two portions when they are attached is selected according to the bone they are intended to fixate. For example, sleeves for fractured femur of cats are designed to have a diameter of between 8-20 mm, or more specifically 10-15 mm.

In some embodiments there may be some overlap between the two sleeve portions, for example the first sleeve portion may surround 270 degrees about the fractured bone and the second sleeve portion may surround 120 degrees about the fractured bone such that there is 30 degrees overlap between the two sleeve potion at each end of the portions.

In some embodiments the fractured bone may have a larger diameter than the diameter of the arc that the two protions of the fixating device define. In such cases, the two portions may encircle only a portion of the periphery of the fractured bone. For example, the arc defined by the two portions of the device may be 1 cm, and the bone may have an average diameter of 1.5 cm. In such case the two portions of the device will encircle about 240 degrees of the periphery of the fractured bone while the remaining 120 degrees of the periphery of the bone will be left exposed.

In one embodiment, the device consists of a first and a second sleeve portions, wherein the combination of the first and second sleeve portions surround 360 degrees of the fractured bone when the device is in use.

The sleeve may comprise a plurality of protrusions attached to or extending from an inner wall of the sleeve so as to press on the fractured bone and fix the bone in place when the device is in use. The protrusions define a space between the bone and the inner wall of the sleeve. Various forms of the protrusions are contemplated. For example, the protrusions may have sharp or blunt tips.

The protrusions may be disposed at various locations as long as they protrude from the inner wall(s) of the sleeve portion(s). The protrusions may be disposed at random locations within the inner wall(s) of the sleeve portion(s). The protrusions may be dispersed along the entire inner wall of the sleeve to press the bone from opposite directions. Alternatively, the protrusions can be dispersed at opposing ends of the sleeve, i.e., proximate the orifices of the sleeve and the protrusions are dispersed in proximity to each of the orifices.

For example, central parts of the sleeve may be free of protrusions while the protrusion are dispersed in proximity to the orifices of the sleeve, such that the sleeve portion that encircles the fracture is free of protrusions. In some embodiments a margin of about 1 mm, 2 mm 3, 5 mm or 1 cm around the fracture is free of protrusions.

The protrusions are made of a rigid or a semi-rigid material being

same or different than the material of the sleeve. The sleeve and/or the protrusions may be made of various materials, such as titanium, titanium alloys (e.g. Ti-6Al-7Nb, Ti-6Al-4V), stainless steel (e.g 316L), cobalt base alloys, bioceramics (e.g. Al₂O₃, ZrO₂), tantalum (in particular tantalum with porous structure), magnesium based alloys, composite materials (e.g. carbon fiber reinforced polymers, CFRP), and polymers (e.g bone, ultrahigh molecular weight polyethylene or UHMWPE. The polymers may be non-resorbable or bioresorbable. The protrusions may be made of a bioinert material as these are the parts of the sleeve which are supposed to come in contact with the bone. The sleeve and/or the protrusion may be made of a biodegradable material such as magnesium based alloys.

The sleeve and sleeve portions may comprise a plurality of apertures that allow for a passage of nutrients and/or cells therethrough to thereby assist in expediting healing of the fractured bone.

Optionally, the combined area of the plurality of apertures includes between 20 to 60% of the area of the sleeve. For example, the combined area of the plurality of apertures incudes at least 10%, at least 20%, at least 30%, at least to 40%, or at least 50%. For example, the combined area of the plurality of apertures incudes no more than 20%, no more than 30%, no more than 40%, no more than 50%, or no more than 60%.

The sleeve portions include an inter-sleeve portion locking mechanism to allow closing the sleeve.

Various locking mechanisms are contemplated, including, for example one or more of screws, straps, wires, zip ties, fasteners, flanges, cerclage, complementary interlocking gooves and protrusions on each portion (jigsaw like) or a combination thereof.

The locking mechanism may be configured to allow closing the sleeve to a given diameter. In one example, the locking mechanism comprises cooperating adjacent edges of the sleeve portions to allow overlap thereof to thereby allow adjustment of the diameter of the sleeve. The locking mechanism may comprise a diameter adjustment fixing fastener comprising an adjustable strap/wire/tie to set the sleeve diameter. The locking mechanism may include fasteners configured to attach overlapping sleeve areas.

Bone growth factors may be added to one or more of the sleeve portions and/or protrusions to assist in expediting healing. The bone growth factors may include bone growth hormones, stem cells, bone growth minerals (e.g., calcium, in particular amorphous calcium, calcium phosphate cements, simvastatin salts, strontium salts, silicone and combinations thereof), and/or bone cells (e.g., osteoblasts, bone marrow concentrate).

The device may optionally comprise an internal film. The film may be disposed in the inner face of the one or more sleeve portions. The firm may comprise bone growth factors, bone growth hormones, stem cells, bone growth minerals (e.g., calcium, in particular amorphous calcium, calcium phosphate cements), and/or bone cells, that can be released to the bone and assist in expediting healing. The film may be attached to the inner side (the side intended to face the fractured bone) of the one or more sleeve portions or provided separately to the provider such as in a kit comprising the sleeve together with the film, allowing the practitioner to attach the film directly to the fractured bone before applying the device to the fractured bone.

Optionally, the one or more elements of the device, e.g., the one or more sleeve portions, may be custom-made to fit the dimensions of the fractured bone. That is, the fractured bone of a patient may be visualized and measured utilizing, for example, a X-ray, CT or MRI scan, and the one or more sleeve portions may be manufactured according to the dimensions (diameter and/or length) of the measured fractured bone and/or the dimensions of the fracture itself. Alternatively, or additionally, the one or more elements of the device, e.g., the one or more sleeve portions, may be manufactured in various fixed sized, so as to fit various patients at different ages and/or genders and/or having different dimensions.

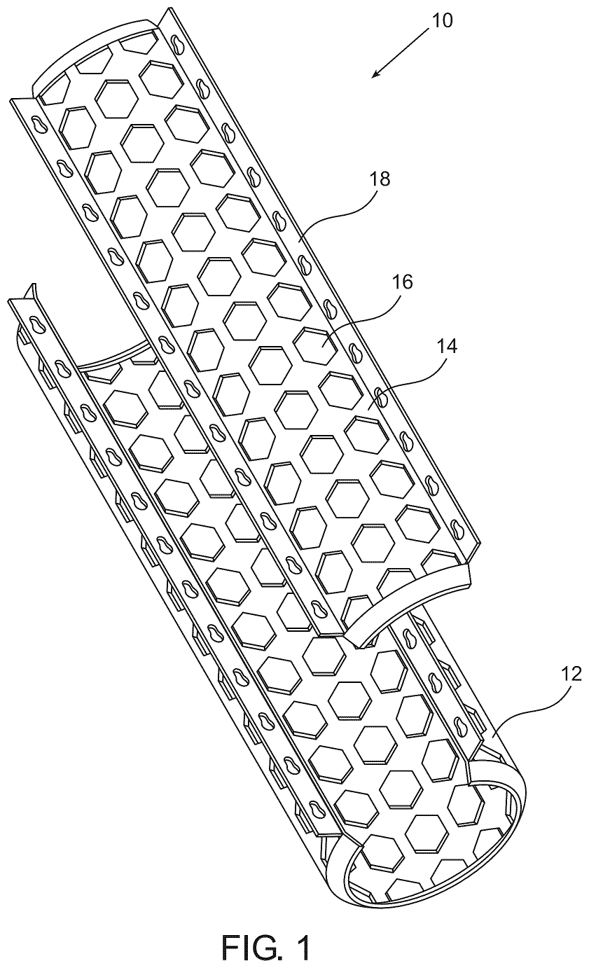

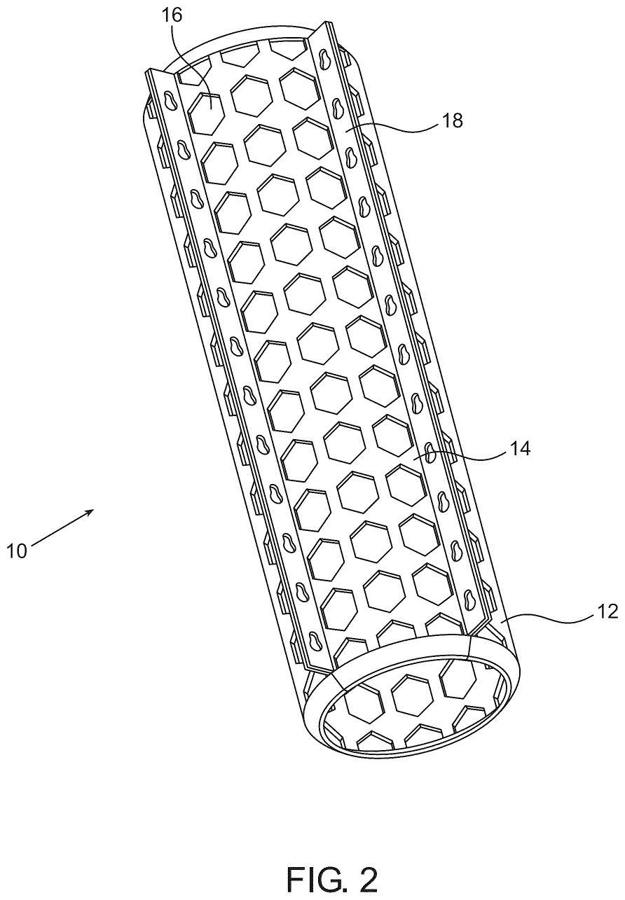

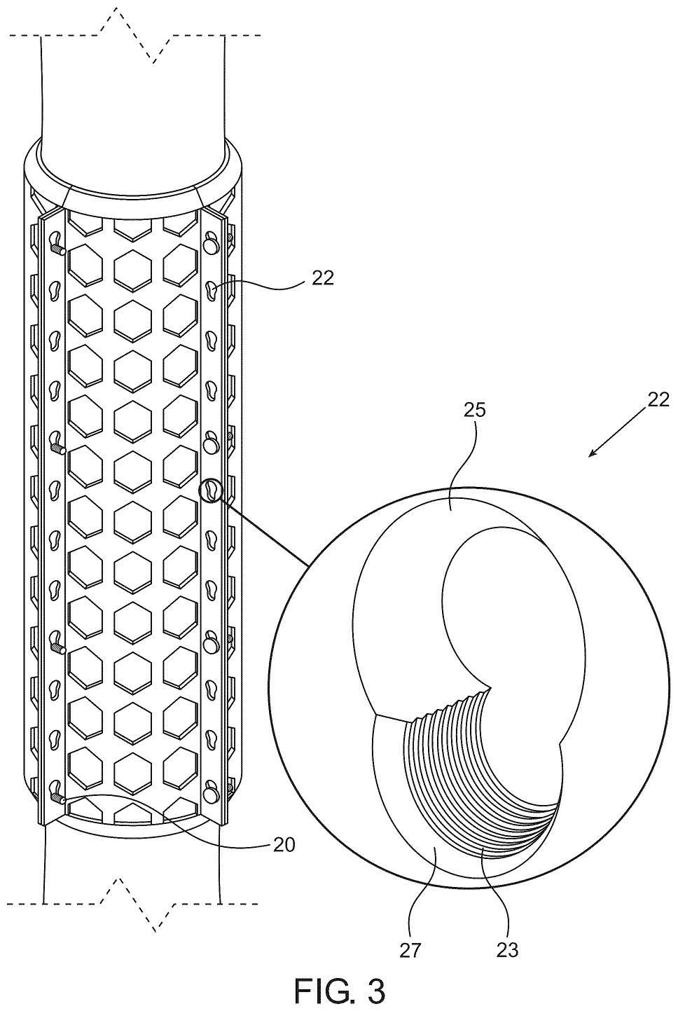

FIG. 1 shows an exemplary bone fracture fixation device 10 including two sleeve portions being a first sleeve portion which constitutes a major sleeve portion 12 and a second minor sleeve portion 14. Together, sleeve portions 12, and 14 form a full sleeve configured to fully or essentially fully surround a fractured bone. Although illustrated as having equal lengths, different lengths of sleeve portions 12 and 14 is also contemplated herein. Each sleeve portion 12, and 14 includes a plurality of apertures 16, typically many apertures, for allowing nutrients flow into the area of the fractured bone to help the healing process. First sleeve portion 12 and second sleeve portion 14 each include a pair of cooperating flanges 18 along edges thereof to allow attaching the sleeve portions to each other (FIG. 2 and FIG. 3, which shows the sleeve portions fastened to each other via fasteners 20 through flange through-holes 22, which may be tear-drop shaped).

Optionally or alternatively, sleeve portions 12, 14 can be attached to each other using straps, cerclage wire or other such fastening mechanisms. Optionally or alternatively, sleeve portions 12, 14 can have edges that, instead of having flanges 18, are configured to overlap so as to allow adjustment of the inner diameter of device 10 so as to provide flexibility in the pressure on the fractured bone and allow adjustment based on the size of the bone. Optionally or alternatively, sleeve portions 12, 14 have a clam-shell configuration with a hinge. Such embodiments have the advantage that the two portions are already connected along the hinge and only require attaching the two portions along one edge. The disproportion between the two sleeve portions allows the insertion of the free edge of the larger portion under the fractured bone followed by the hinged edged while leaving the smaller portion out of the open wound and closing the clam shell only after the free edge of the larger sleeve portion emerges from the other side of the fractured bone. In some embodiments, due to spatial constraints, the sleeve portions are hinge-less.

FIG. 3 shows an exemplary enlarged hole 22 which may include a wider portion 25 that allows insertion therethrough of a fastener 20. Contiguous to the wider hole portion 25 is a narrower portion 27 with an internal thread 23 which allows to fasten a fastener, here screw 20, thereto, and thereby secure the first sleeve portion 12 to the second sleeve portion 14. The flanges 18 may protrude outwardly from the sleeve portions 12 and 14 in angles that allow full attachment between the sleeve portions 12 and 14. Flanges 18 may include several holes 22 along their length to enable attachment between the sleeve portions 12 and 14 at various positions. The plurality of holes provides a translational degree of freedom for matching the two sleeve portions one with respect to the other (i.e., the last two holes of first portion 12 can face the pendulum two holes of the second portion 14 of the sleeve. This freedom may allow the implementation of the device amid spatial constraints in-situ which require it, i.e., in clinical situations allowing little room to maneuver the device.

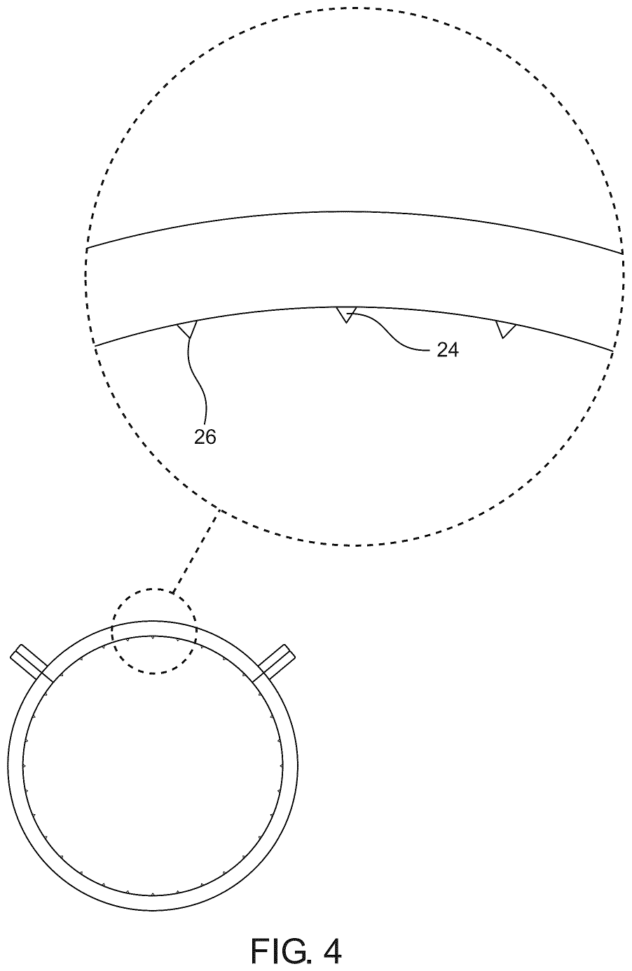

FIG. 4 shows an end view of device 10 in a closed (bone fixing/setting) position with an enlargement of a section thereof illustrating a plurality of inwardly-facing bone-fixation protrusions 24, at least some of which are intended for interfacing with the fractured bone. Inwardly-facing bone-fixation protrusions 24 are configured and dimensioned so as to fixate device 10 to the fractured bone and to provide a space between the fractured bone and the sleeve portions 12, 14.

Protrusions 24 may be integrally formed with sleeve portions 12, 14 or affixed thereto. Protrusions 24 may have tips 26 that can be sharp. Rounded or flat protrusions 24 are also contemplated herein and may be used. Protrusions 24 may include bone growth factors/growth-hormones/calcium/stem cells to be released to the fractured bone to expedite healing.

Sleeve portions 12, 14 and/or bone fixation protrusions 24 can be made of a semi-rigid material to allow for better contact with and micro-movement of the fractured bone. Optionally or alternatively, device 10 may include three or more sleeve portions, one of the sleeve portions defines at least 180 degrees of the device surrounding the fractured bone.

During placement of the device 10 at the bone fracture site, it is preferable that major sleeve portion 12 is distal to the surgical incision/underneath the fractured bone and that minor sleeve portion 14 is proximal to the surgical incision/above the fractured bone. Nevertheless, major sleeve portion 12 may alternatively be positioned proximal to the surgical incision/above the fractured bone and minor sleeve portion 14 may be distal to the surgical incision/underneath the fractured bone.

It is to be understood that by “distal to/underneath” it is also meant to placing major sleeve portion 12 farther from the surgical incision. It is to be further understood that by “proximal to/above” it is also meant to placing minor sleeve portion 14 closer to the surgical incision.

A method of fixating a fractured bone of a human or non-human patient will now be described. The method includes a first step (a) of providing access for the bone fracture fixation device to the fractured bone; a second step (b) of placing a sleeve portion at least partially around, the fractured bone; a third step (c) of placing one or more additional sleeve portions around the fractured bone, so as to fully or essentially fully surround at least a portion of the fractured bone; and a fourth step (d) of fastening the sleeve portions to each other. Optionally, prior to step (a), the fractured bone is visualized and measured, and the one or more sleeve portions is manufactured to fit the dimensions (length and/or diameter) of the fractured bone.

In the first step, providing access for the bone fracture fixation device to the fractured bone, may include providing an incision in the skin area, as customary in the art, which is sufficient for inserting the portions of the bone fracture fixation device inside the organ containing the fractured bone. The size of the incision may need to be sufficient for manual insertion of the portions of the bone fracture fixation device. The incision may be minimal, as customary in minimal invasive procedures, as to allow the insertion of a minimal invasive means, such as an arthroscope. In such cases the arthroscope is designed to bring the portions of the bone fracture fixation device to the target location around the fractured bone. The bone fracture fixation device may be portioned to several portions allowing the portions to be compatible with the dimensions of the arthroscope.

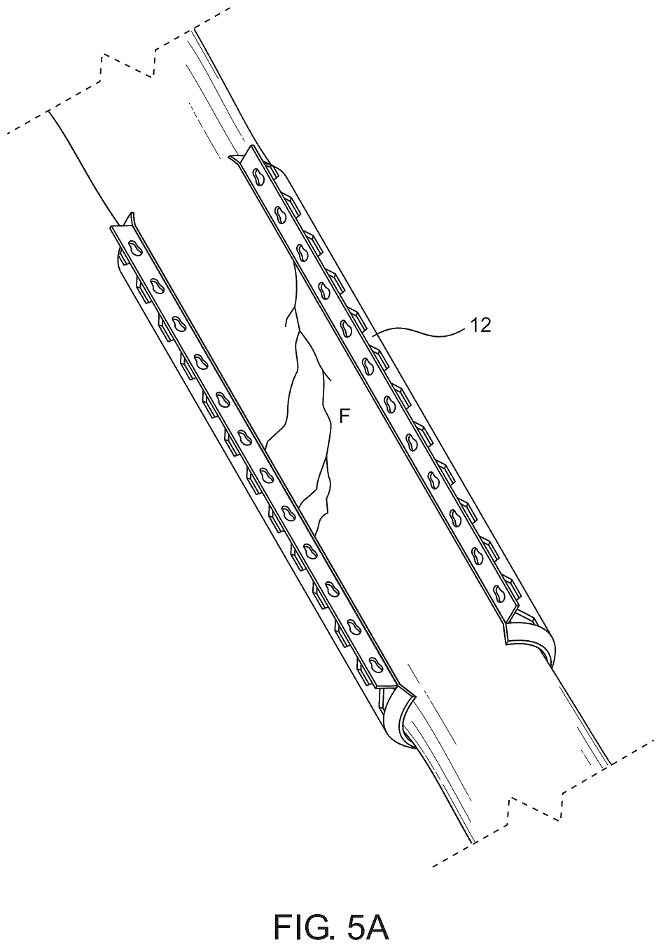

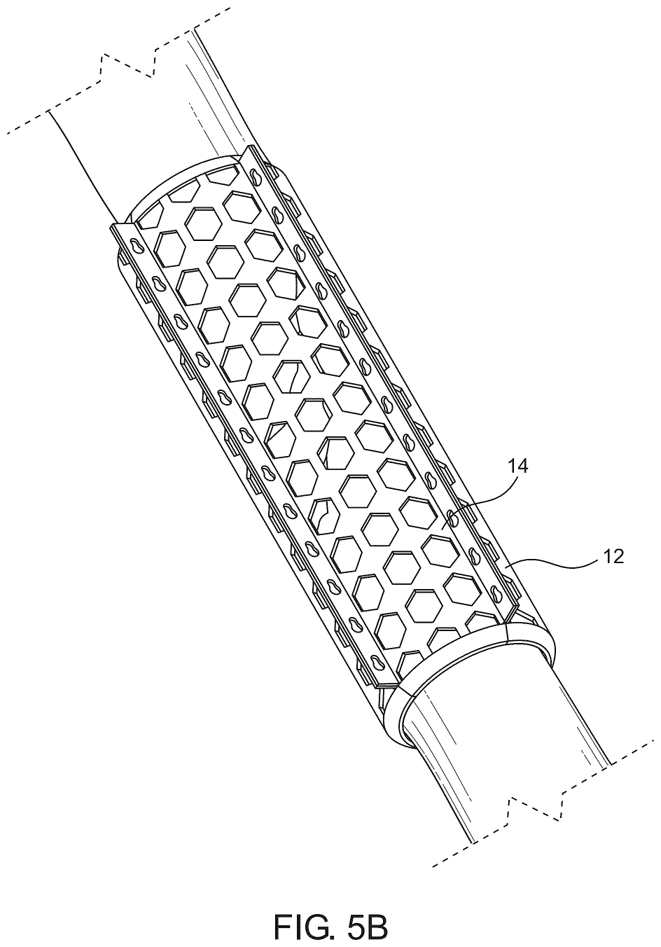

The method may, in the second step, include first surrounding the fractured bone to an extent of greater than 180 degrees by one of the sleeve portions, and in particular wherein that sleeve portion is placed underneath the fractured bone, during step (b). In the context of this invention underneath the fractured bone means the side of the bone that is distal from the surgical incision. As exemplified in FIG. 5A first sleeve portion 12 may be firstly placed around the fractured bone such that the fractured bone area F faces the surgeon. As such, first sleeve portion 12 being the large portion, is inserted below the fractured bone and placed between the bone and the tissues underlying the bone, usually by sliding the first portion about the fractured bone from one side till it engulfs the fractured bone and appears from the other side. As exemplified in FIG. 5B, during step (c) the second minor sleeve portion 14 is placed on top of the first sleeve portion 12 closer to the surgical cut in a manner that the sleeve portions 12 and 14 complement each other and fully surround or essentially fully surround the fractured bone. The disproportion between the two portions of the sleeve enables the insertion of the major first portion 12 as described above with minimally moving or stretching neighboring tissues or organs as compared to a device made of two equal portions. The method may also include adjusting the diameter of the resultant sleeve to a given diameter about the fractured bone.

The above-mentioned step (d) may entail adjusting the diameter of the resultant sleeve so as to interface at least some of the protrusions with the fractured bone. Step (d) may also entail using a locking mechanism configured to allow fastening using one or more of screws, straps, wires, zip ties, fasteners, flanges, cerclage or a combination thereof. Step (d) may optionally or alternatively entail overlapping adjacent edges of the sleeve portions to thereby allow adjustment of the diameter of the sleeve and fixing thereof. Optionally or alternatively, after step (d), the method may further include removing the device from around the fractured bone.

Above-mentioned step (a) may include surgically cutting tissue adjacent the bone fracture.

The method may include using two sleeve portions that are predesigned and manufactured to adapt the dimensions of the fractured bone.

Or, the method may include using off the shelf device with sleeves having fixed dimensions that can match the diameter of the fractured bone. The method may include using the device in veterinary purposes, alternatively or in addition to use for humans.

Above-mentioned step (b) may include placing the first sleeve portion so that it is located distally or proximally from the surgical cut made during a surgery to insert the sleeve portions in-vivo (inside the body of the patient).

The method of the invention may dramatically reduce the duration of the procedure. The method may reduce the duration of the procedure by 25%, 30%, 35%, 40%, 45% or even 50% when compared to the surgical procedures for fixation of fractured bones available in the prior art. For example, surgery of a cat having a traverse proximal simple fracture of the femur may consume no more than 20, 15, or even 10 minutes.

In a further aspect the invention provides the use of a bone fracture fixation device of the invention in treatment of a patient in need according to the method of the invention. The patient may be human or non-human, i.e the use may be for medical and/or for veterinary treatments.

Each of the following terms: ‘includes’, ‘including’, ‘has’, ‘having’, ‘comprises’, and ‘comprising’, and their linguistic equivalents, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The term ‘consisting essentially of’ as used herein means that the scope of the claim is limited to the specified elements and those that do not materially affect the basic and novel characteristic(s) of the claimed device and materials.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

All ranges disclosed herein include the endpoints. The use of the term “or” shall be construed to mean “and/or” unless the specific context indicates otherwise.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### Example 1

An exemplary procedure according to the invention is given below as can be evidenced with X-ray and stills images.

A cat being injured with a central transverse fracture of the femure; can be treated by:
1. Cat is given anesthesia of isoflorane
2. The skin above the fractured femur can be sanitized followed by 4 cm long incision to provide access for the bone fracture fixation device to the fractured bone;
3. Fracture fragments are fixed together;
4. First sleeve portion of 200 degrees arc of a 1 cm diameter and 10.4 mm length can be positioned around the fractured bone on the side distal from the cut;
5. A second sleeve portion of 160 degrees arc of a 1 cm diameter can be positioned around the fractured bone on the side being proximal to the cut, to fully surround the portion of the fractured bone containing the fracture and a margin of a few centimeters above and below the fracture or in the case were the bone diameter is larger than the 1 cm diameter of the two joint arcs, then a portion of the fractured bone may be left exposed;
6. The sleeve portions can be fastened to each other by four cerclages;
7. Routine work-up ends the surgery;
8. The cat can be given dipyrone and amoxicillin for a week.

The duration of the procedure may last for about 10 to 15 minutes. The cat may put weight on the foot 2 days after surgery, and walk freely 5 days after surgery. This example may demonstrate the feasibility of the device and procedure for expedite recovery and short procedure time for the surgeon.

1-42. (canceled)

43. A bone fracture fixation device for setting a fractured bone, the device comprising:
a sleeve having measures to fully or essentially fully surround at least a segment of the fractured bone including the fracture, the sleeve comprising:
at least two sleeve portions, wherein a first sleeve portion having measures to surround above 180 degrees about the fractured bone, and a second sleeve portion and optionally one or more additional sleeve portions complementing the first sleeve portion to thereby fully or essentially fully surround the fractured bone when the device is in use.

44. The device of claim 43, wherein the first sleeve portion surrounds more than 210 degrees, more than 230 degrees, more than 250 degrees, or more than 270 degrees of the fractured bone when the device is in use.

45. The device of claim 43, comprising a first and a second sleeve portions, wherein the first and second sleeve portions surround 360 degrees about the fractured bone when the device is in use.

46. The device of claim 43, wherein the sleeve further comprises a plurality of protrusions attached to or extending from an inner wall of the sleeve to press on the fractured bone and fix the bone in place when the device is in use.

47. The device of claim 46, wherein the protrusions are characterized by at least one of: (i) have sharp or blunt tips; (ii) are dispersed along the entire inner wall of the sleeve; (iii) are dispersed on the two or more portions of the sleeve; (iv) define a space between the bone and the sleeve; and (v) are made of a rigid or a semi-rigid material being same or different than the material of the sleeve.

48. The device of claim 46 wherein the sleeve comprises two orifices through which the fractured bone passes when the device is in use and the protrusions are dispersed in proximity to each of the orifices.

49. The device according to claim 48, wherein a central portion of the inner wall of the sleeve that is proximal to the fracture is free of protrusions.

50. The device of claim 43, wherein the sleeve portions comprise at least one of: (i) a plurality of apertures to allow passage of nutrients and/or cells therethrough to assist in expediting healing of the fractured bone; (ii) an inter-sleeve portion locking mechanism to allow closing the sleeve; and (iii) bone growth factors, bone growth hormones, bone growth minerals (e.g., calcium), bone cells, stem cells or mixtures thereof, that are releasable to the bone and assist in expediting healing.

51. The device of claim 50, wherein the combined area of the plurality of apertures consists between 20 to 60% of the area of the sleeve.

52. The device of claim 50, wherein the locking mechanism is characterized by at least one of: (i) comprises one or more of screws, straps, wires, zip ties, fasteners, flanges, cerclage or a combination thereof; (ii) configured to allow closing or at least partially closing the sleeve to a given diameter; (iv) comprises cooperating adjacent edges of the sleeve portions to allow overlap thereof to thereby allow adjustment of the diameter of the sleeve; and (v) comprises a diameter adjustment fixing fastener comprising an adjustable strap/wire/tie to set the sleeve diameter; or through-sleeve fasteners configured to attach overlapping sleeve areas.

53. The device of claim 43, further comprising a film disposed in the inner face of the one or more sleeve portions, the film comprising bone growth factors, bone growth hormones, bone growth minerals, bone cells, stem cells, or mixtures thereof that can be released to the bone and assist in expediting healing.

54. The device of claim 43, wherein the two sleeve portions have different lengths.

55. A method of fixating a fractured bone of a human or non-human patient, the method comprising:
(a) providing access for the bone fracture fixation device of claim 43 to the fractured bone;
(b) placing the first sleeve portion at least partially around the fractured bone;
(c) placing the second sleeve portion and optionally one or more additional sleeve portions around the fractured bone, so as to fully or essentially fully surround at least a portion of the fractured bone; and
(d) fastening the sleeve portions to each other.

56. The method of claim 55, wherein the first sleeve portion is dimensioned to surround above 180 degrees of the fractured bone and wherein placing the first sleeve portion is characterized by at least one of (i) being performed prior to placing the second sleeve portion and the optionally one or more additional sleeve portions, so as to allow full surrounding of the fractured bone; and (ii) placing the first sleeve portion underneath the fractured bone.

57. The method of claim 55, further comprising at least one of: (i) adjusting the diameter of the resultant sleeve to a given diameter; and (ii) placing a film in the side of the sleeve portions to face the fractured bone or directly on the fractured bone, wherein the film comprising bone growth factors, bone growth hormones, bone growth minerals (e.g., calcium), stem cells and/or bone cells, that can be released to the bone and assist in expediting healing.

58. The method of claim 55, wherein step (d) entails at least one of (i) adjusting the diameter of the resultant sleeve so as to interface at least some of the protrusions with the fractured bone; (ii) using a locking mechanism configured to allow fastening using one or more of screws, straps, wires, zip ties, fasteners, flanges, cerclage or a combination thereof; and (iii) overlapping adjacent edges of the sleeve portions to thereby allow adjustment of the diameter of the sleeve and fixing thereof.

59. The method of claim 55, wherein after step (d), the method further comprises removing the device from around the fractured bone.

60. The method of claim 55, wherein step (a) comprises surgical cutting tissue adjacent the bone fracture.

61. The method of claim 55, wherein the at least two sleeve portions are predesigned and custom-made to adapt the dimensions of the fractured bone according to imaging diagnosis.

62. The method of claim 55, wherein step (b) comprises at least one of: (i) placing the first sleeve portion underneath the fractured bone; (ii) placing the first sleeve portion so that it is located distally or proximally from the surgical incision; (iii) placing the first sleeve portion so that it is located proximally from the surgical incision.
